(19) United States
(12) Patent Application Publication
(10) Pub. No.: US 2010/0069811 A1
(43) Pub. Date: Mar. 18, 2010

(54) ANTI-MICROBIAL BAMBOO FIBERS AND FABRICS

(76) Inventor: Rohitashwa PODDAR, Mumbai (IN)

Correspondence Address: KENYON & KENYON LLP, ONE BROADWAY, NEW YORK, NY, 10004, US

(21) Appl. No.: 12/233,113

(22) Filed: Sep. 18, 2008

(51) Int. Cl. A61F 13/00; A61K 36/58; A61B 19/08

(52) U.S. Cl. 602/43; 424/761; 428/221; 128/849

(57) ABSTRACT

The invention provides a method for modifying bamboo fibers, yarns, and textiles, by contacting them with a solution of water-soluble components of the Neem tree, azadirachta indica. The modified materials exhibit improved anti-microbial properties.

### FIELD OF THE INVENTION

The invention relates to the fields of textile manufacturing and anti-microbial technology.

### BACKGROUND OF THE INVENTION

Bamboo fiber is a form of regenerated cellulose fiber, typically produced through one of two solvent spinning processes. In the viscose process, bamboo-derived cellulose is dispersed in an aqueous solution of caustic soda (ca. 20%), to obtain alkali cellulose. The dispersion so formed (the “steep”) generally has a solids content of 2-6%. Excess liquid is removed in roll or belt presses to isolate the alkali cellulose, which is then aged by exposure air to achieve a degree of oxidative degradation, reducing the chain length of the cellulose. The alkali cellulose is then reacted with carbon disulfide to form a cellulose xanthate. The cellulose xanthate is subsequently dissolved in dilute caustic soda, forming the solution known as viscose. The viscose is forced through spinnerets into an acidic spin bath, which coagulates the viscose to form fiber and converts the cellulose xanthate back to cellulose.

An alternative is the Lyocell process, in which bamboo-derived cellulose pulp is dissolved in N-methylmorpholine N-oxide, creating a solution which is forced through spinnerets into a water bath. After the fibers are recovered and washed, the solvent is retrieved from the water, purified, and recycled. Since there are few byproducts and little waste, and because bamboo may be grown in quantity with minimal use of fertilizers and pesticides, the overall process is considered ecologically friendly.

Bamboo fiber so produced is comparable to rayon fiber marketed under a variety of trade names, and may be used in the same products. Staple fibers are used in apparel items such as denim, chino, underwear, casual and fine clothing, and bath towels. The fiber can be blended with a variety of other fibers such as silk, cotton, rayon, polyester, linen, nylon, and wool. The fibers are also used in specialty papers and medical dressings.

One of the more unusual properties of bamboo fiber is its anti-microbial activity. Growth of bacteria and fungi is inhibited by contact with a fabric woven from bamboo fibers, and as a result there is a growing interest and commerce in items such as towels, socks, underwear, and medical dressings made from bamboo fiber.

The problem of nosocomial infections in hospitals is a particularly acute problem, given the weakened condition of many patients and the tendency of nosocomial organisms to exhibit multiple drug resistance. Infection control procedures in hospitals include the treatment of exposed surfaces with bactericidal compositions, and the frequent cleaning and/or sterilization of hospital garments, masks, bedding, and dressings. It is known to impregnate textiles with rubber, polymer, and bactericidal compositions so as to inhibit the growth and spread of microorganisms; see for example U.S. Pat. Nos. 5,849,311, 5,069,907, 6,034,010, and references therein. All such textile items can be rendered more inhospitable to bacterial and fungal growth by manufacturing them with bamboo fiber.

Improvements upon the anti-microbial activity of bamboo fibers and fabrics would lead to improved performance of products where anti-microbial activity is desirable, and the present invention is directed to such an improvement.

### BRIEF DESCRIPTION OF THE INVENTION

The present inventors have discovered that treatment of bamboo fiber with one or more of the chemical substances (components) present in a decoction containing water-soluble components of azadirachta indica (Neem tree) results in fabrics and textiles having enhanced anti-microbial activity. Surprisingly, there is no detectable residue of azadirachta indica components, nor are there detectable azadirachta indica components leaching from the materials. When samples of bamboo fiber textiles, modified by the process of the invention, are tested for anti-microbial activity, bacterial growth is inhibited at the point of contact, but no clearing zone is observed around the textile samples. The anti-microbial activity persists through numerous washings of the fabrics. Accordingly, it appears that the treatment of bamboo fibers by the methods of the invention results in a chemical and/or physical modification of the fibers. Bamboo fibers thus modified by contact with one or more of the components present in a decoction of azadirachta indica exhibit improved anti-microbial properties, and are particularly useful for the manufacture of clothing, particularly shirts, pants, underwear and socks, and for the manufacture of hospital garments, bed linens, towels, and dressings.

A decoction of azadirachta indica suitable for use in the invention may be produced by a process including the steps of (a) suspending crushed and dried leaves of azadirachta indica in water, and (b) removing insoluble material from the resulting suspension to produce a solution. Preferably, the process further comprises evaporating the solution to yield a residue, referred to herein as azadirachta indica solids, which material can be conveniently stored, transported, and weighed and used in a reproducible manner.

Preferably, the bamboo fiber is spun into yarn and woven into a textile, and all chemical processing steps such as desizing and bleaching are carried out prior to contact of the textile with a decoction of azadirachta indica. Contacting with azadirachta indica decoction can be carried out simultaneously with dyeing, by adding the decoction to the dye solution, so that the bamboo fibers are dyed while they are in contact with the decoction of azadirachta indica. Alternatively, the bamboo fiber is contacted with the decoction immediately after dyeing, preferably by adding the decoction directly to the dye bath once the dye has set.

### DETAILED DESCRIPTION OF THE INVENTION

As used herein, “decoction of azadirachta indica” or “decoction” refers to an aqueous solution of water-soluble materials, which are extracted with water from azadirachta indica. The solid residue resulting from evaporation of the decoction is referred to herein as “azadirachta indica solids”. The individual chemical compounds present in the decoction and in the azadirachta indica solids are referred to as “water-soluble components”.

The enhanced bamboo fibers of the invention are useful for making textile items such as clothing, underwear, socks, and towels. They are particularly useful for textile products used in a hospital setting, such as bed linens, surgical garments, drapes, and dressings. Surgical garments include, but are not limited to, gloves, gowns, facemasks, hats, hoods, aprons, shirts and pants, shoe covers, and booties, whether worn by patients or medical professionals. Surgical drapes are the sheets of fabric that are draped over a patient, creating a sterile and absorbent field around the site of surgery. Dressings include bandages, absorbent pads, and other textile-based items that are used to cover and protect a wound.

The invention provides a method of enhancing the anti-microbial properties of bamboo fibers, comprising the step of contacting the fibers with one or more of the water-soluble components that are present in a decoction of azadirachta indica. Preferably, the method comprises contacting the fibers with a decoction of azadirachta indica. The decoction may be directly prepared by water extraction of azadirachta indica plant material, but is preferably prepared by dissolution of a measured quantity of azadirachta indica solids. The bamboo fibers may be in the form of a thread, spun yarn, or woven fabric, and may be blended with other fibers such as cotton, wool, polyester, and the like. Blended fiber compositions are preferably at least 70% bamboo fiber by weight. Preferably, the bamboo fibers are woven into a textile, processed, and dyed prior to contacting the fibers with the decoction of azadirachta indica.

The decoction of azadirachta indica is preferably produced by combining crushed and dried leaves of azadirachta indica with water, and removing insoluble material from the resulting mixture to produce a solution. Preferably, the mixture is maintained at a temperature between 10° C. and 40° C., for 5-7 hours, before the solids are removed. Preferably, the solution is then evaporated to yield azadirachta indica solids, which may be conveniently stored, transported, and measured, and can be re-constituted into an aqueous solution prior to use.

A preferred method of re-constitution comprises preparing a solution of about 1% to about 5.5% azadirachta indica solids in water. More preferably, a solution of about 1% to about 3% azadirachra indica solids is prepared, and concentrated by evaporation to yield a concentration of about 4% to about 5.5%. A solution containing about 5% azadirachra indica solids is preferred as the working solution.

The methods of the invention may be applied to undyed bamboo fibers. In other embodiments, bamboo fibers may be dyed and then contacted with a decoction of azadirachta indica. In a preferred embodiment, the fibers are dyed and contacted with the decoction simultaneously, by the simple expedient of adding the decoction to a dye solution prior to carrying out the dyeing process. Dyeing may be by any of the continuous or discontinuous methods known in the art, as may be appropriate to the particular fabric weave and the dye or dyes being used. Suitable methods include but are not limited to vat dyeing methods, padding methods, and jet-dyeing, for example as disclosed in U.S. Pat. No. 6,044,509 and references therein.

A representative embodiment of the invention comprises (a) adding to a dye bath an amount of the decoction of azadirachta indica, and (b) contacting the bamboo fibers with the dye bath. An alternative embodiment comprises (a) immersing the bamboo fibers in a dye bath; (b) allowing the bamboo fibers to become dyed; and (c) adding a decoction of azadirachta indica. The amount of decoction is preferably sufficient to provide a final concentration of about 0.005 to about 0.05 grams of azadirachta indica solids per liter, more preferably about 0.01 grams of azadirachta indica solids per liter. In a preferred embodiment, which avoids the introduction of additional processing steps, a decoction of azadirachta indica (preferably sufficient to provide a final concentration of about 0.01 grams of azadirachta indica solids per liter) is added to the dye solution prior to contacting the fibers with the dye. Modification of the fiber by the soluble azadirachta indica component(s) takes place within the time required for setting of the dye.

In preferred embodiments, the dye bath is a metal-free dye bath. Preferred dyes are cellulose-reactive dyes such as are commercially available under the Cibacron™ F, Remazol™ and Procion™ marks. It is also preferred that the bamboo fibers are treated by formaldehyde-free processes.

The invention provides thread, yarns, and textiles comprising bamboo fibers made by the processes described above, as well as surgical garments, drapes, bed linens, or dressings comprising the textiles of the invention. The invention also provides non-surgical garments such as towels, and bed linens, underwear, socks, and bath and facial towels, comprising these textiles.

### EXAMPLES

Methods for the preparation, processing, and weaving of bamboo fiber are known to those skilled in the art, and are not detailed here. See for example “BambroTex Technical Guidance Documents”, China Bamboo Textile Co. Ltd. (2003).

1. Desizing: Bamboo fiber was prepared by the viscose process, formed by conventional means into yarn, and woven into fabric on an air-jet loom. The thread count of samples ranged from 10/1 Nm to 40/1 Nm. (Single-fiber threads, having weights from 10 meters to 40 meters per gram.) Samples of the woven fabric were desized by impregnating with a solution of desizing enzyme (Ciba Tinozyme™ L40) (4.0 g/l), wetting agent (Ciba Ultravon™ EL) (2.0 g/l), and cracking agent (Ciba Invatex™ ED) (0.5 g/l), and maintained at 60° C. for 12 hours. Treatment agents were removed with a hot water wash.

2. Bleaching: The fabric was scoured and bleached at the same time with a solution of hydrogen peroxide (20 g/l), sodium hydroxide (10 g/l), wetting agent (Ciba Ultravon™ PRE) (4.0 g/l), stabilizer (Ciba Clarite™ MAX) (2.0 g/l), and a surfactant/dispersant/cracking agent (Ciba Invatex™ MD) (1.0 g/l), with a 20-minute steaming (98° C.) and hot wash off.

3. Finishing: The desized, scoured and bleached fabric was neutralized to pH 5 with acetic acid (2 g/l), and washed with a softener (Clariant Sandolub™ HD) (10 g/l).

4. Dyeing and treatment with decoction of azadirachta indica: The fabric was dyed by the cold pad-batch method with vinyl sulfone reactive dyes. Reactive Yellow 24 (Remazol™ Yellow RTN) (1.9 g/l), Reactive Blue 21 (Remazol™ Turquoise Blue G) (6.5 g/l), and Reactive Blue 220 (Remazol™ Blue BB) (1.2 g/l) were employed with sodium carbonate (20 g/l) as auxiliary, an alkali-compatible additive combining anti-foam, surfactant and leveling agents (Ciba Albatex™ FFC-01) (2.0 g/l), and sufficient azadirachta indica decoction (5% azadirachta indica solids) to yield 0.01 g/l of azadirachta indica solids. After a dwell time of 3-24 hr, the fabric was washed and dried conventionally.

5. Anti-microbial testing: Samples of treated and untreated bamboo fiber fabric, approximately 24-25 mm wide, were laid across streaks of Staphylococcus aureus (ATCC 6538) growing on solid medium. Three control and three experimental samples were tested, each sample was cut into five pieces and each piece laid across a streak of S. aureus culture. After incubation for 24 hr, fabric samples were removed and the plates examined. Bacterial growth was observed under all of the control samples, and clearing was observed under all treated samples. Cleared zones were smaller than or commensurate with the measured sample widths, and no zone of clearance extending beyond the contact area was noted. Results are presented in Table 1. Complete inhibition of Candida albicans ATCC 10231 has also been observed.

TABLE 1
S. aureus ATCC #6538
Inhibition By Fabric Samples

| | | | Growth | Total | |
|---|---|---|---|---|---|
| | | Sample | Under Sample | clearing | Zone size |
| Sample ID | Streak # | Width | (Y/N) | (mm) | (mm) |
| #0001 Rep 1 | 1 | 25.06 | N | 25.06 | (No zone) |
| | 2 | 24.80 | N | 24.76 | (No zone) |
| | 3 | 24.37 | N | 24.00 | (No zone) |
| | 4 | 25.27 | N | 25.27 | (No zone) |
| | 5 | 24.66 | N | 18.27 | (No zone) |
| #0001 Rep 2 | 1 | 26.28 | N | 25.55 | (No zone) |
| | 2 | 26.03 | N | 23.08 | (No zone) |
| | 3 | 24.82 | N | 19.92 | (No zone) |
| | 4 | 23.97 | N | 20.43 | (No zone) |
| | 5 | 24.19 | N | 8.62 | (No zone) |
| #0001 Rep 3 | 1 | 23.04 | N | 18.85 | (No zone) |
| | 2 | 23.36 | N | 19.70 | (No zone) |
| | 3 | 24.76 | N | 22.36 | (No zone) |
| | 4 | 25.13 | N | 20.64 | (No zone) |
| | 5 | 25.46 | N | 20.89 | (No zone) |

| | | | | |
|---|---|---|---|---|
| Average | 24.75 | N | 21.16 | (No zone) |

We claim:

1. A method of enhancing the anti-microbial properties of bamboo fibers, comprising the step of contacting the fibers with one or more of the water-soluble components present in a decoction of azadirachta indica.

2. The method of claim 1, comprising the step of contacting the fibers with a decoction of azadirachta indica.

3. The method of claim 2, wherein the bamboo fibers are in a form selected from the group consisting of thread, spun yarn, and woven fabric.

4. The method of claim 2, wherein the bamboo fibers are processed and dyed prior to contacting the fibers with the decoction of azadirachta indica.

5. The method of claim 3, wherein the bamboo fibers are processed and dyed prior to contacting the fibers with the decoction of azadirachta indica.

6. The method of claim 2, wherein the bamboo fibers are dyed while they are in contact with the decoction of azadirachta indica.

7. The method of claim 3, wherein the bamboo fibers are dyed while they are in contact with the decoction of azadirachta indica.

8. The method of claim 2 or claim 3, wherein the decoction of azadirachta indica is produced by a process comprising the steps of:
(a) suspending crushed and dried leaves of azadirachta indica in water;
(b) removing insoluble material from the resulting suspension to produce a solution;
(c) evaporating the solution to yield azadirachta indica solids; and
(d) preparing about a solution of about 1% to about 5.5% azadirachta indica solids in water.

9. The method of claim 8, wherein the decoction of azadirachta indica is produced by a process comprising the steps of:
(a) suspending crushed and dried leaves of azadirachta indica in water;
(b) removing insoluble material from the resulting suspension to produce a solution;
(c) evaporating the solution to yield azadirachta indica solids;
(d) preparing about a solution of about 1% to about 3% azadirachta indica solids in water; and
(e) concentrating the solution to produce a concentrate containing from about 4% to about 5.5% azadirachta indica solids.

10. The method of claim 9, wherein the concentrate contains from about 5% azadirachta indica solids.

11. The method of claim 8, comprising the steps of
(a) immersing the bamboo fibers in a dye bath;
(b) allowing the bamboo fibers to become dyed; and
(c) adding an amount of the decoction of azadirachta indica sufficient to provide a concentration of 0.01 grams of azadirachta indica solids per liter.

12. The method of claim 8, comprising the steps of
(a) adding to a dye bath an amount of the decoction of azadirachta indica sufficient to provide a concentration of 0.01 grams of azadirachta indica solids per liter, and
(b) contacting the bamboo fibers with the dye bath.

13. The method of claim 8, wherein the dye bath is a metal-free dye bath.

14. The method of claim 8, wherein the bamboo fibers are prepared by a formaldehyde-free process.

15. A thread comprising bamboo fibers enhanced by the process of claim 8.

16. A yarn comprising thread enhanced by the process of claim 8.

17. A textile comprising thread or yarn enhanced by the process of claim 8.

18. A surgical garment, drape, or dressing comprising the textile of claim 17.

19. A non-surgical garment, towel, or bed linen comprising the textile of claim 17.
